(19) United States
(12) Patent Application Publication
(10) Pub. No.: US 2018/0317732 A1
(43) Pub. Date: Nov. 8, 2018

(54) CLEANING ARTICLE HAVING AN ABSORBENT BASE MATERIAL

(71) Applicant: CMC Consumer Medical Care GmbH, Sontheim an der Brenz (DE)

(72) Inventors: Rainer MANGOLD, Herbrechtingen (DE); Angela Roempp, Duernau (DE); Maria Arkhipova, Neu-Ulm (DE)

(21) Appl. No.: 15/773,218

(22) PCT Filed: Nov. 11, 2016
(86) PCT No.: PCT/EP2016/077482

§ 371 Date: May 3, 2018

(30) Foreign Application Priority Data
Nov. 12, 2015 (EP) 15194393.3

(51) Int. Cl. A47L 13/16; D06N 7/00; A61K 8/02; A61Q 19/10

(52) U.S. Cl. CPC A47L 13/16; D06N 7/0092; A61K 8/0208; A61K 2800/28; D06N 2209/106; D06N 2211/08; D06N 2211/24; A61Q 19/10

(57) ABSTRACT

The invention relates to a cleaning article (100) having an absorbent base material formed by a flat material (101), with a first (102) and a second side surface, wherein a coating (112) is provided on at least the first side surface (102) and confers to said first side surface (102) of the cleaning article (100) a higher frictional force than that of an uncoated first side surface (102), wherein the coating (112) comprises a plurality of coating lines (114) and, when an imaginary grid network (200) consisting of lines extending parallel, equidistantly and vertically to one another and with an edge length of 7 mm for each grid quadrant (206) is laid over the first side surface (102) of the cleaning article (100), said first side surface (102) has a first degree of coating coverage of at least 15% in the region of at least 5 grid quadrants (208) per 25 cm², and a second degree of coating coverage of no more than 8% in the region of at least 5 grid quadrants (210) per 25 cm².

The invention relates to a cleaning article which has an absorbent base material and is formed by a flat material having a first side face and a second side face, wherein a coating which has been provided on at least the first side face imparts elevated cleaning power to the first side face of the cleaning article compared to an uncoated first side face. The prior art discloses a multitude of cleaning articles, for instance for surface cleaning, especially for domestic cleaning, and also for skin cleaning, especially for face cleaning.

For example, DE 20 2005 014 927 U1 describes a cotton pad for cleansing and exfoliation of the skin, wherein an application of binder on at least one outer side of the cleaning article in at least one subregion and at least one further subregion free of binder application have been provided. The binder can be applied in various patterns, for example wavy lines, grids, dots or fishscale patterns. The patterns envisaged are comparatively small pattern elements.

Likewise known from WO 03/104544 A1 is a scrubbing wipe article having a structured application of coating, wherein the pattern density of the applied coating is comparatively uniform.

Proceeding from this prior art, it is an object of the present invention to provide a cleaning article which, with simultaneously good cleaning action, provides sufficient absorption areas for constituents to be removed and, if the cleaning article is one for skin cleansing, especially face cleansing, is perceived as being pleasant to the skin or, if the cleaning article is one for surface cleaning, especially in the domestic sector, it nevertheless also has compensating regions suitable, for example, for polishing, soft wiping.

The invention achieves this object by a cleaning article having the features of claim 1, in which the coating comprises a multitude of coating lines and wherein, when an imaginary grid composed of parallel, equidistant lines running at right angles to one another with an edge length of 7 mm of each grid square is placed over the first side face of the cleaning article, the first side face has a first degree of coverage of coating of at least 15% in the region of at least 5 grid squares per 25 cm² and a second degree of coverage of coating of at most 8% in the region of at least 5 grid squares per 25 cm².

A cleaning article in the context of the invention shall be understood to mean not only cosmetic cleansing articles such as, in particular, cosmetic pads, cosmetic tissues, but also cleaning articles for surface cleaning, such as, in particular, cleaning cloths, for example for domestic purposes, especially for cleaning of dishware, but also for cleaning of surfaces, preferably in the kitchen or sanitary sector, and for glass and window cleaning etc.

A grid of parallel, equidistant lines running at right angles to one another shall be understood to mean an arrangement of lines in which an array of parallel, equidistant lines is intersected by a second array of parallel, equidistant lines to form right angles. In the grid thus formed, onto which the cleaning article can be placed as desired, meaning that there is no preferential direction of the grid, at least 5 grid squares per 25 cm² should then have a first degree of coverage of coating of at least 15% and hence a high degree of coverage, and at least 5 grid squares per 25 cm² a second degree of coverage of coating of at most 8% and hence a comparatively low degree of coverage. By definition, only those grid squares that are completely within the reach of the cleaning article are to be used for the determination. Grid squares that are intersected by the boundary line (edge) of the cleaning article are disregarded for the consideration of the latter squares to be counted.

The stipulation “per 25 cm²” is understood as a reference parameter. In the evaluation, the entire side face of the cleaning article is considered. In the case of cleaning articles having smaller or greater dimensions other than 25 cm², the number of grid squares having first and second degrees of coverage ascertained on the first side face of the cleaning article is converted correspondingly with standardization to 25 cm².

The degree of coverage is understood to mean the region covered by the coating within a grid square based on the area of the grid square. For the evaluation of the degree of coverage of the coating in relation to the respective grid squares, it is possible to use any desired evaluation methods, for example computer-assisted graphic and drawing programs.

In this way, it is possible to provide a cleaning article which, owing to regions having a relatively high degree of coverage by virtue of a high coating density, especially a high coating density of coating lines, has a good exfoliating effect or cleaning effect for stains. On the other hand, a cleaning article of this kind, however, owing to a likewise large number of regions having only a low degree of coverage, has sufficient absorption or uptake area for the components released from the surface to be cleaned, for example soil particles, skin particles, makeup or moisture. Absorption or uptake areas of this kind can then also simultaneously be soft to the skin if, for example, a cosmetic tissue or cosmetic pad is to be realized, or else have a certain softness with respect to hard surfaces if, for example, a domestic cloth is to be realized. This is especially also achieved in that the edge length off the grid squares at 7 mm is large compared to the small-scale patterns known in the prior art, such that large coherent cleaning areas and large coherent absorption areas are created in each case.

In a preferred embodiment, it may be the case that the first side face has a first degree of coverage of at least 15% in the region of at least 8, particularly at least 10, more particularly at least 15, more particularly at least 20, more particularly at most 35, more particularly at most 30, more particularly at most 25 grid squares per 25 cm².

In addition, it may also preferably be the case that, in the case of the configuration of the coating with at least 10, especially at least 15, grid squares per 25 cm² having the first degree of coverage of at least 15%, at least 5 grid squares of these have a degree of coverage of at least 20%.

In this way, the advantages mentioned can be even better realized.

More particularly, the grid squares having the first degree of coverage of at least 15% have an upper limit of the degree of coverage of particularly at most 50%, more particularly at most 40%, more particularly at most 30%, more particularly at most 25%.

Especially advantageously, the coating has a coverage quotient, ascertained from the number of grid squares per 25 cm² of the second degree of coverage of not more than 8% divided by the number of grid squares per 25 cm² of the first degree of coverage of at least 15%, of at least 0.1, particularly at least 0.2, more particularly at least 0.3, more particularly at least 0.4, more particularly at most 3.0, more particularly at most 2.5, more particularly at most 2.0, more particularly at most 1.8, more particularly at most 1.6. By virtue of this relative arrangement of regions with a comparatively low degree of coverage relative to regions with a comparatively high degree of coverage, it is possible to take account of the different end uses of the cleaning article. More particularly, cleaning articles having a coating having a coverage quotient of 0.1-1.0 are usable for a desirable stronger cleaning or exfoliating effect; by contrast, cleaning articles having a coating quotient of 1.1-3.0 can be geared to the aspect of more balanced friction/cleaning characteristics relative to absorption areas.

For the provision of a cleaning article having regions for cleaning/frictional action and simultaneously having regions for absorption areas, the coating of the first side face of the cleaning article can especially advantageously have at least one region, particularly at least 2 regions, more particularly a multitude of regions in which at least 2, particularly at least 3, particularly at least 4, more particularly at most 30, more particularly at most 25, more particularly at most 20, grid squares of the first degree of coverage of at least 15% are in a directly adjacent arrangement, and particularly at least one region, particularly at least 2 regions, more particularly a multitude of regions in which at least 2, particularly at least 3, more particularly at least 4, grid squares of the second degree of coverage of not more than 8% are in a directly adjacent arrangement.

“Directly adjacent” grid squares are understood to mean those joined via a common edge or a common vertex.

The coating of the cleaning article comprises coating lines. Complete coating of the cleaning article in the sense of a continuous, uninterrupted application over the full area is not the intention. As well as coating in the form of coating lines, however, it is also possible to provide partial areal coatings or dotted coatings.

Such coatings other than the linear coatings are to be taken into account in accordance with the invention in the determination of the degree of coverage. In other words, the respective degrees of coverage are found via the coatings disposed in the respective grid squares, irrespective of the configuration thereof.

However, exclusively flat or exclusively homogeneously distributed dotted coatings are not in accordance with the invention.

A linear coating is especially understood to mean an element in which a line width of at least 0.2 mm is provided and the line has a length at least five times the line width.

The linear coatings may in principle comprise both straight lines and curved lines, and also intersecting straight or curved lines. The line may in principle be configured either in continuous or interrupted form at least in regions, provided that the line as such remains clearly apparent. In other words, dotted, dashed-and-dotted or dotted coating lines are also conceivable in the context of the present invention. More particularly, the sites of interruption must not be longer than ten times, particularly not longer than eight times, particularly not longer than six times, particularly not longer than four times, the line width of this line adjacent to this site of interruption.

In addition, it may be the case that the coating comprises or has been formed from a multitude of individual patterns. The individual patterns especially comprise coating lines or have especially been formed from coating lines. Individual patterns shall be understood to mean those patterns in the form of open or closed patterns. Open patterns are those patterns in which the start of the line has no contact with the end of the line, and closed patterns are those in which the start and end of a line can no longer be ascertained since they are joined to one another. Furthermore, only those patterns that cannot be reduced to a single point shall be considered as individual patterns according to the invention. In other words, an individual pattern must be more than one point, and individual patterns are preferably those in which the coating line does not extend exclusively in just one vector direction as a straight line, but those in which the linear pattern has curves and/or kinks.

As well as these individual patterns, however, it is also possible for further coatings to be provided, for example in the form of dotted or else areal patterns.

It may be the case here that the individual patterns are arranged in a mutually discrete manner and in such a way that they cannot be imaged by one or more continuous, especially regularly continuous, coating lines running continuously from a first edge of the first side face to an opposite second edge of the first side face. What is achieved in this way is that the coating lines do not run merely in a preferential direction.

Edges of the cleaning article are understood to mean all edges or borders of the cleaning article and boundaries thereof.

Mutually discrete individual patterns are those that are either completely separated from one another or else individual patterns that also touch, intersect with and/or overlap with one another. The individual pattern, in spite of touching, intersecting and/or overlapping, is still recognizable as an individual pattern from its areal extent defined by the direction set by the coating line. Individual patterns are also understood to mean groups of patterns which are especially composed of at least two identical and/or different pattern elements. Arrangements regarded as groups of patterns are especially those in which at least two pattern elements are arranged alongside and in contact with one another, and/or especially also groups of patterns in which a first pattern element at least partly, especially completely, surrounds or encircles a second or further pattern element for example concentric arrangements, especially of circles, ovals or triangles or other polygons, or concentric geometric figures of any kind that are in contact at one point or a line section. The coating may also comprise combinations of such groups of patterns.

It will be apparent that the individual patterns formed from coating lines in particular are at least partly, preferably completely, surrounded by an uncoated region and/or else encompass an uncoated region and at least partly, preferably completely, encircle this uncoated region.

In a particularly preferred embodiment, it may be the case that at least one individual pattern is formed on at least the first side face such that a section of this coating runs at right angles to any direction running across the area of the cleaning article or, in the case of a curved cleaning article, to any tangential direction across the cleaning article. This means that, for any possible direction in the cleaning article, provided that the cleaning article lies flat, there exists a section or region in the coating that runs at right angles to any direction in the plane. By virtue of the configuration of the linear coating with a curve, it is possible to achieve better distribution of the forces in different directions. The exfoliating effect and abrasive effect in respect of stains can be improved in this way. In a further particularly preferred embodiment, the section may be dotted, in which case an imaginary tangent applied to this point always runs at right angles to any direction in the cleaning article. More preferably, at least 20%, especially at least 40%, especially at least 50%, especially at least 60%, especially at least 80%, especially 100%, of the individual patterns have at least one section that runs at right angles to any direction of the area of the cleaning article. Especially in at least 20%, especially at least 40%, especially at least 50%, especially at least 60%, especially at least 80%, especially 100%, of the individual patterns, this at least one section is in dotted form, and an imaginary tangent applied thereto runs at right angles to any direction in the area of the cleaning article.

The linear coatings provided on the at least one first side face of the cleaning article may have identical or different geometric shapes and may especially be configured with identical or different measurements/dimensions. More particularly, it is also possible to combine linear patterns with areal or dotted patterns.

In addition, it is preferably possible for at least one individual pattern to be formed as a group of patterns comprising at least two pattern elements formed from coating lines. More preferably, at least 20%, especially at least 40%, especially at least 50%, especially at least 60%, especially at least 80%, of the individual patterns are formed from a group of patterns. More particularly, every individual pattern is formed from a multitude of pattern elements. The group of patterns may be formed, for example, from internal and external pattern elements and/or may be formed to give an overall pattern of adjoining pattern elements or further pattern elements arranged, for example, alongside and in contact with one another. Particular preference may be given to the arrangement of a group of patterns in such a way that one pattern element of an individual pattern encircles at least regions of, but preferably entirely encircles, a second pattern element or further pattern elements. Encircling shall especially also be understood to mean that the lines are in contact with one another at least in regions or run parallel to one another. More particularly, the group of patterns of an individual pattern may be configured such that a first pattern element is arranged alongside and in contact with a second pattern element or further pattern elements. Combinations of groups of patterns are also conceivable. Overall, the abrasive effect can be further improved by groups of patterns.

It may especially be the case here that the individual patterns are surrounded by an uncoated outer region having a geometric form different than the geometric form of the individual patterns.

What is achieved in this case is that, unlike in grid or strip patterns, there is no preferential direction, but instead an area for absorption; all-round drapability can be provided equally efficiently. Similar effects can especially also be achieved in that various coatings, for example linear coatings, are combined with other coatings, such as dotted or linear coatings.

It is particularly preferable that at least one individual pattern on the at least one first side face is encircled all round by an uncoated outer region. It is further particularly preferable that a multitude of individual patterns in each cleaning article that have been applied on the first side face is surrounded on all sides by an uncoated outer region. Especially preferably, all individual patterns in the cleaning article are surrounded by an uncoated outer region. What can be achieved in this way is that, firstly, by virtue of the linear coatings of the individual patterns in particular, the cleaning and abrasive effect of the cleaning article can be increased in the respective regions. On the other hand, the desired properties of the base material, for example absorption properties, and also tactile properties of the cleaning article are simultaneously maintained.

According to the desired degree of the abrasive properties and desired visual features and configuration, the multitude of coatings may be applied in a regular repeating pattern or may be arranged in an irregular manner.

It is especially the case here that the coating of the cleaning article and here especially the first side face comprises essentially over its entire extent, i.e. not just specific regions such as the middle or outer region of the cleaning article. It is therefore preferably the case that the coating extends over the entire at least first side face of the cleaning article, in which case it is possible, according to the pattern provided, for individual regions of the cleaning article to vary, for example the middle or edge region, with regard to the pattern density in the context of the invention defined. It is also conceivable to apply different patterns in some regions of the cleaning article than in other regions. For example, a different coating in the middle region than in the edge region may be provided.

In addition, it may be the case that the coating may be provided in homogeneous or inhomogeneous distribution across the area of the cleaning article.

Preferably, just the first side face is coated. In another configuration, however, both side faces may also be coated.

More preferably, the cleaning article may have a degree of coverage by the coating of at least 6%, particularly at least 8%, more particularly at least 10%, more particularly at least 15%, more particularly at least 20%, and particularly at most 50%, more particularly at most 40%, more particularly at most 30%, and more particularly at most 25%. In this way, a good cleaning effect of the cleaning article is achieved, and the desired properties of the base material, for example absorption capacities, are not altered too significantly, but maintained.

If the coating on the cleaning article comprises closed patterns, it may be the case that the individual patterns occupy a total area proportion of at least 10%, particularly at least 20%, more particularly at least 30%, more particularly at least 40%, and particularly not more than 70%, more particularly not more than 60%, more particularly not more than 50%, of the at least first side face. The area of an individual pattern is understood here to mean the region enclosed by the outer coating lines (including the coating lines); thus, the inner uncoated regions of the individual pattern or, in the case of execution as a group of patterns, the corresponding areas of the individual pattern elements are also taken into account. If individual patterns have closed pattern regions, and further open, i.e. not intrinsically closed, coating lines extend away from them, these free appended coating lines are not taken into account in the calculation of the area of the individual pattern. Over the areas covered by the individual patterns, it is possible for sufficient abrasive properties to be achieved while maintaining the properties inherent to the base material, such as softness and absorption properties.

An individual pattern preferably has an area having a distance between the outer coating lines of at least 0.3 cm, preferably at least 0.5 cm, more preferably at least 0.7 cm, more preferably at least 1.0 cm, more preferably at least 1.5 cm, more preferably at least 2 cm, more preferably at most 5 cm, more preferably at most 4 cm, more preferably at most 3 cm. The distance, which may be a diameter for example, is understood to mean the distance between the coating lines describing or delimiting an individual pattern that are the furthest apart in the distal direction in each case. This involves measurement at the outer edge of the coating line, i.e. including the line width thereof.

An individual pattern, including the encircling coating lines, preferably has an area of at least 0.2 cm², more preferably of at least 0.5 cm², more preferably of at least 1.0 cm², more preferably of at least 1.5 cm², more preferably of at most 10.0 cm², more preferably of at most 8.0 cm², more preferably of at most 6.0 cm².

The coating features may be different in terms of their geometric form and/or their dimensions or the same. It is possible here for the different properties of the cleaning article, such as degree of coverage, abrasiveness, absorption properties, to be taken into account and to be implemented via the establishment of the coating patterns.

Particular preference is given to coating patterns having curved or rounded regions, since these, in the use state, enable better ergonomic adaptation to the surface to be cleaned and a more pleasant feel.

The line width of the coating line may be at least 0.2 mm, particularly at least 0.4 mm, particularly at least 0.5 mm and more particularly at least 0.6 mm. At the same time, the line width should preferably be at most 2.0 mm, more particularly at most 1.6 mm, more particularly at most 1.2 mm, more particularly at most 1.0 mm. The length of the coating line in relation to the line width is especially to amount to at least 5 times, preferably at least 6 times, further preferably at least 8 times and further preferably at least 10 times the line width.

The height of the coating lines and of any further coatings provided is to be at least 0.1 mm, particularly at least 0.2 mm. The height of the coating line is particularly to be at most 0.8 mm, more particularly at most 0.6 mm and more particularly at most 0.4 mm. The measurement of the height can be ascertained with a microscope having appropriate magnification, specifically as the difference between an averaged upper top side of the base material and the upper edge of the coating line.

Preferably, the entire coating is raised above the upper top side of the base material.

With these preferred heights of the coating lines and of any further coatings provided, tactile effects with an unpleasant feel are advantageously avoided.

The basis weight of the coating may be at least 5 g/m², particularly at least 10 g/m², more particularly at least 15 g/m² and more particularly at least 20 g/m². At the upper end, the basis weight is preferably limited to 50 g/m², more particularly to not more than 30 g/m² and more particularly to not more than 25 g/m².

The coating is especially polymer-based. More particularly, the coating is based on a polymer taken from the group comprising PE (polyethylene), PP (polypropylene), APAO (amorphous poly-alpha-olefins), EVA (ethylene-vinyl acetate), EVAC (ethylene-vinyl acetate polymers), PA (polyamides), TPE-O (olefin-based thermoplastic elastomers), TPE-V (crosslinked olefin-based thermoplastic elastomers), TPE-E (thermoplastic copolyesters), TPE-U (urethane-based thermoplastic elastomers), TPE-A (thermoplastic copolyamides, e.g. PEBA), TPE-S (thermoplastic styrene block copolymers), for example HSBC (hydrogenated styrene block copolymers), SEBS (styrene-ethylene-butadiene-styrene polymers), SBS (styrene-butadiene-styrene), SEPS (styrene-ethylene-propylene-styrene) or a combination of one or more of the polymers mentioned.

The coating is preferably homogeneous in terms of its composition. Preferably, no abrasive particles are added to the coating.

Useful materials for the coating preferably include materials having a Shore A hardness of at least 30, particularly of at least 40, particularly at least 50, more particularly at least 60, and especially of not more than 95, more particularly of not more than 90, more particularly not more than 80, more particularly not more than 70. Shore A hardness is a material index for elastomers and polymers. Shore A hardness is determined by the method which follows.

Methods of Determining Shore a Hardness:

Shore A hardness is a measure of the resistance of a material to the penetration of a body of a particular shape and under a defined spring force. In the units of Shore hardness, the value of 0 reports the lowest hardness and the value of 100 the greatest.

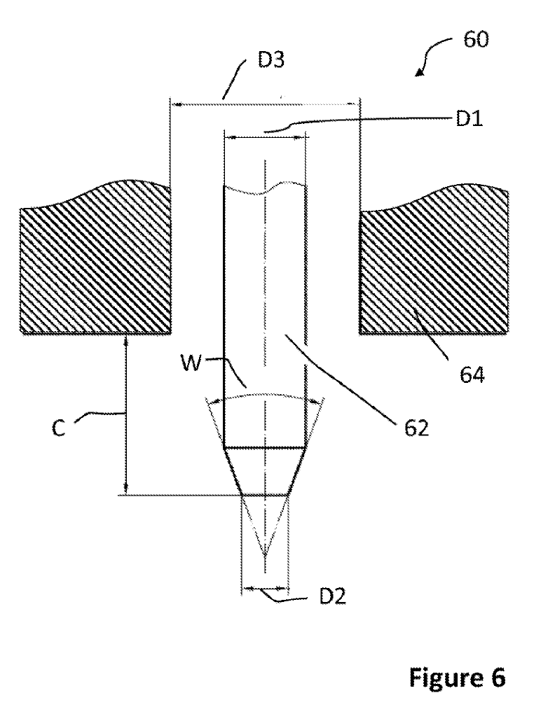

The measurement is in accordance with the standards DIN 53505:2000-08 and ISO 868:2003(E). A Shore A hardness tester is used here. A Shore A hardness tester of this kind, which is shown schematically in FIG. 6 with reference numeral 60, uses a sprung indentation body having the geometry of a frustocone. The indentation body 62 made of steel has a diameter D1 of 1.25±0.15 mm which merges into a lower frustocone having a lower area having a diameter D2 of 0.79±0.01 mm with an angle of inclination W of 35°±0.25°. The distance C between the lower edge of a pressure foot 64 and the lower area of the indentation body is 2.5±0.02 mm. The indentation body is centered within the pressure foot 64 with a recess having a diameter D3 of 3±0.5 mm.

The test is to be conducted on mechanically non-prestressed specimens. For the test, the test specimen is already to have polymerized or vulcanized to completion for 16 hours. The test is conducted under standard conditions at 23±2° C. and 50±2% air humidity. The test specimens and the instruments have been correspondingly conditioned for at least 1 hour.

The test specimens need dimensions that allow measurements at least 12 mm from each edge, and at the same time have a sufficiently plane-parallel contact surface in order that the pressure foot can contact the test specimen over an area of radius at least 6 mm around the tip of the indentation body. Specimens having a material thickness of at least 4 mm are required. In the case of small thicknesses, the test specimens may be composed of multiple thinner layers. Measurements are made at at least 5 different points on each test specimen, where the distance from the edges of the test specimen is at least 12 mm. The distance between the measurement sites is to be at least 6 mm. The contact weight of the indentation body is 1 kg.

The measurement time is 3 seconds; in other words, the hardness is read off 3 seconds after the contact surface of the test instrument and the test specimen come into contact.

The coating lines are preferably applied by means of a roller that has engraving corresponding to the pattern (sum total of the individual patterns).

The first side face having the coating may have a dynamic coefficient of friction measured in accordance with ASTM D 1894-01 of at least 0.2, particularly at least 0.3, more particularly at least 0.4, more particularly at least 0.5, more particularly at least 0.6, more particularly at least 0.7, where the maximum values to be achieved are particularly at most 1.5, particularly at most 1.2, more particularly at most 1.0. In this way, sufficient abrasiveness and cleaning power is generated.

Test for Determination of the Dynamic Coefficient of Sliding Friction:

In the present context, the sliding characteristics of cleaning articles of the invention with coating are to be ascertained. In this case, the first side face of the cleaning article provided with the coating is drawn over a standardized surface. The force A of sliding friction that occurs here is to be measured, and this is then to be used to determine the dynamic coefficient of sliding friction. The test method is based on ASTM D 1894-01, for determination of the friction characteristics of polymer films.

The test specimens have to be conditioned under standard climatic conditions at 23° C.±2° C. and 50%±2% air humidity for at least 2 hours. The samples must not be kinked, folded or scratched; other alterations and contaminations should be avoided. The same applies to the test plate made of steel. The test method should likewise be conducted under standard conditions (23° C.±2° C., 50%±2%).

A test specimen of dimensions 50×50 mm or diameter 50 mm is punched out of the cleaning article with coating or out of a corresponding material in roll form, and secured on a rubbing block in a fold-free manner. However, the material in roll form is exactly the same material from which the cleaning articles according to the invention have been punched or cut.

The rubbing block has a base area of edge length 63 mm×63 mm, i.e. a contact base area of 40 cm², and a mass of 200 g±5 g. It is secured by means of a thread (with no intrinsic strain) to the load cell of a tensile tester according to DIN 51 221 Class 1. A tensile tester of this kind is the Zwick Roell 2010 from Zwick GmbH&Co. KG, 89079 Ulm, Germany.

The additional instrument consisting of the sample table and rubbing block according to DIN EN ISO 8295:2014 is likewise supplied by Zwick. The rubbing block with the test specimen is placed cautiously onto a defined material, a smooth polished steel plate (DIN EN 1939: 2003-12). 15 seconds after the application of the rubbing block, the test is started. The test speed is 150 mm/min, both for the actual measurement distance of 130 mm and for the pre- and post-measurement distance each of 10 mm. For the determination of the dynamic coefficient of sliding friction p, only the force profile of the measurement distance of 130 mm is used. The test is conducted for at least five test specimens. A mean x and the standard deviation s are reported with rounding to two post-decimal places. The dynamic coefficient of sliding friction is found from the quotient of the force A of sliding friction thus determined, expressed in grams (g), divided by the force of 200 g exerted by the rubbing block.

The cleaning article, with regard to the base material, may be in single- or multi-ply form and may especially comprise a fiber material, especially a nonwoven material. Nonwoven materials preferably include natural cellulose-based fibers, for example, cotton or viscose, or synthetic fibers, for example polyesters, preferably polyester bicomponent fibers or polyester microfibers, polypropylene or mixtures thereof. The nonwoven material may especially be a nonwoven made from continuous fibers or preferably a nonwoven made from staple fibers.

The base material, in the case of a voluminous configuration of the cleaning article, especially if it comprises cottonwool-like products, such as cosmetic pads in particular, especially also in the case of multi-ply base material, has a base ply having a basis weight preferably of at least 80 g/m², more preferably of at least 100 g/m², more preferably of at least 120 g/m², more preferably of at least 150 g/m², more preferably of at least 200 g/m², more preferably of at most 300 g/m², more preferably of at most 280 g/m². In the case of a more tissue-like configuration of the cleaning article, the basis weight may preferably be at most 100 g/m², preferably at least 30 g/m² and more preferably at least 40 g/m².

The thickness of the cleaning article, in this case including the coating on the first side face or optionally a coating on the second side face, is 0.3-5 mm, preferably 1-3 mm. In the case of a tissue-like configuration of the cleaning article, the thickness is preferably 0.3-1.5 mm; in the case of a voluminous configuration of the cleaning article, especially if it comprises more cottonwool-like products, such as cosmetic pads in particular, the thickness is preferably 1.0-5 mm. The determination of the thickness of the cleaning article (including the coating) is conducted using a specific measurement pressure of 0.5 kPa over a sensor area of 25 cm². More particularly, a Schroder DMT thickness meter can be used. For the rest, the thickness is determined in accordance with DIN EN ISO 9073-2: 1995.

Especially preferably, the cleaning article has the coating of the invention only on the first side face. In this way, the second coating-free side face of the cleaning article can be used in further acts of use that precede and/or follow the cleaning or exfoliating effect of the first side face.

Especially preferably, the cleaning article has a coating on the first and second side faces. More particularly, the cleaning article has the coating of the invention on both side faces. In this way, the cleaning article can be used by the user for more intensive cleaning operations and also in a simpler manner without regard to the orientation of the cleaning article.

The cleaning article is preferably usable for cleansing of the skin, especially face cleansing, for instance as a cosmetic pad or cosmetic tissue, such as, more particularly, as an exfoliating pad or exfoliating tissue. As well as cosmetic cleaning articles, the cleaning article is also usable for cleaning of hard surfaces. More particularly, the cleaning article is usable as a cleaning cloth, for example for domestic purposes, especially for dishware cleaning, but also for surface cleaning, preferably in the kitchen or sanitary sector, and for glass and window cleaning.

The cleaning article is preferably a disposable product. In principle, however, cleaning articles, for example in the domestic sector, that can be washed or cleaned are also conceivable.

In the present manner, it is possible to provide a cleaning article having particularly favorable properties in terms of abrasiveness and absorptivity.

Further features and details and advantages of the invention will be apparent from the drawings and description of the cleaning article of the invention which follows. The figures show:

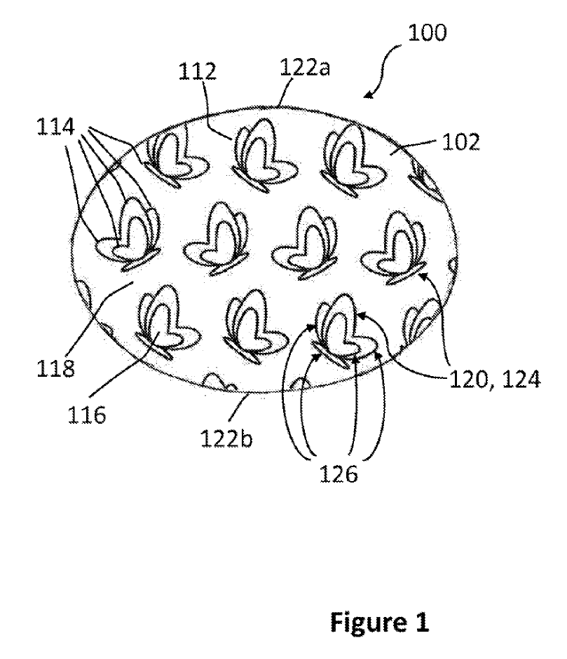

FIG. 1 a diagram of a first side face of a cleaning article of the invention,

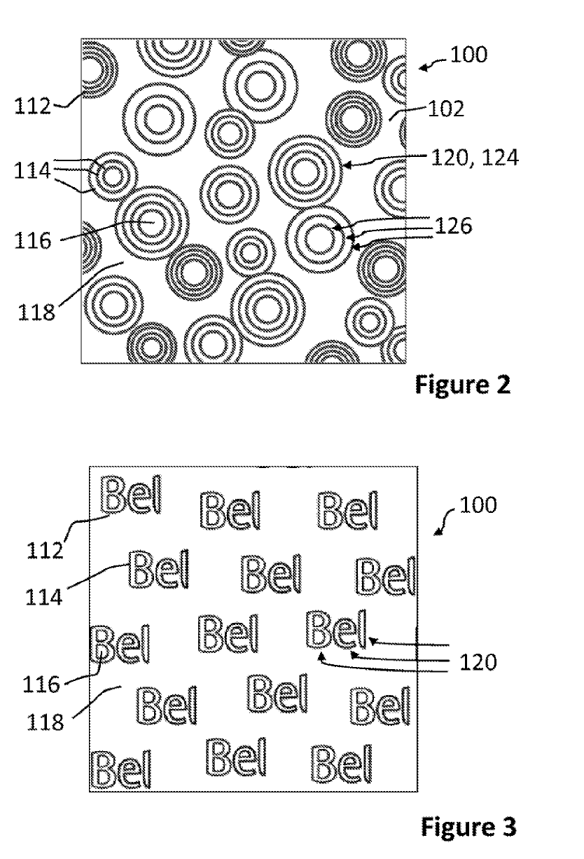

FIG. 2 a diagram of a first side face of a further cleaning article of the invention,

FIG. 3 a diagram of a first side face of a further cleaning article of the invention,

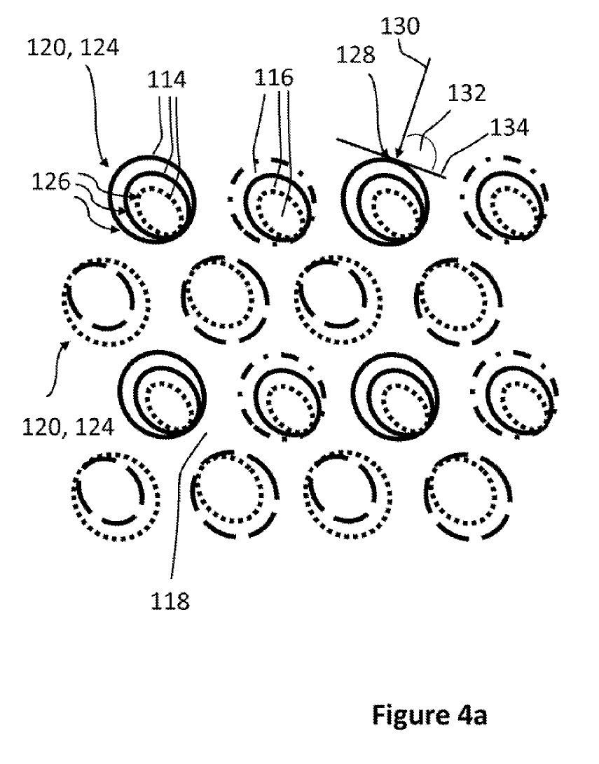

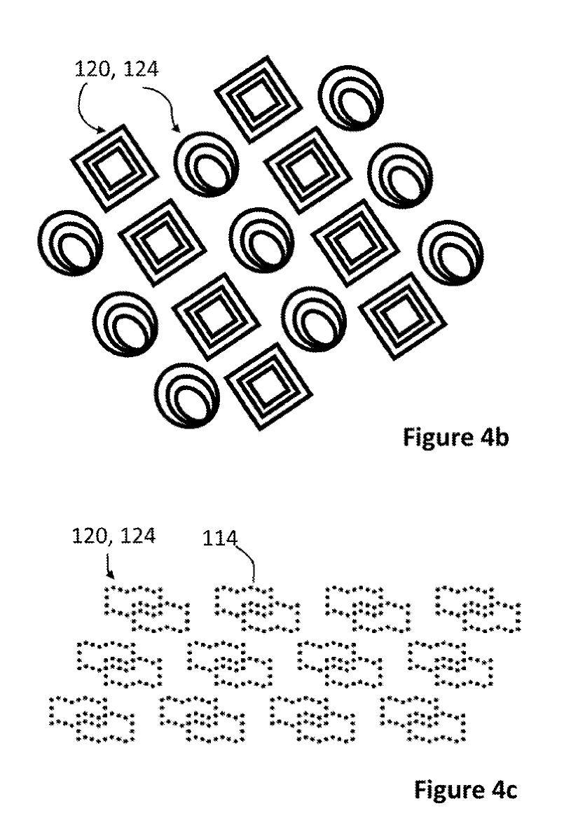

FIGS. 4a-c) various individual patterns of the coating,

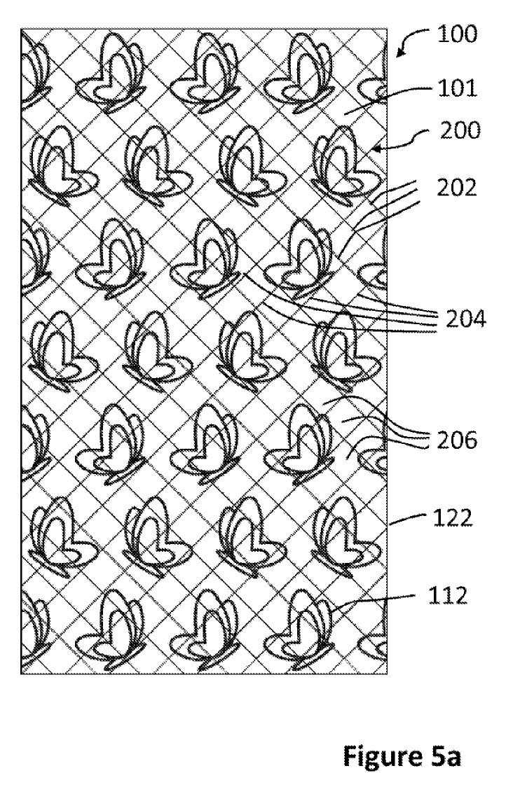

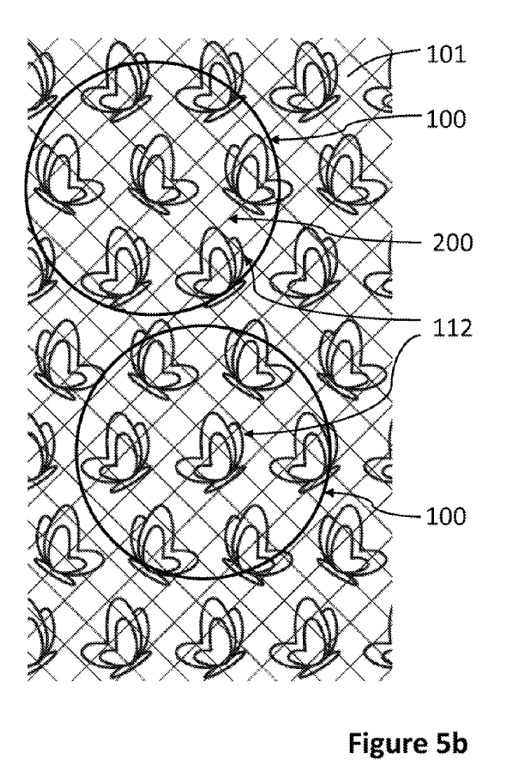

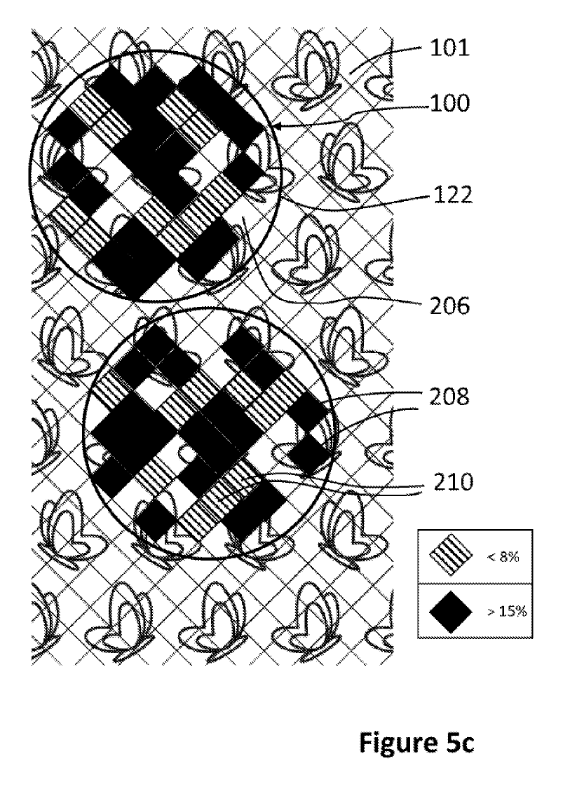

FIGS. 5a-c) a diagram of the determination of the grid squares and

FIG. 6 a schematic diagram, not to scale, of a detail of a Shore A hardness measuring instrument.

FIG. 1 shows a top view of the first side face 102 of a cleaning article 100 of the invention, in the configuration of a cosmetic pad, wherein the first side face 102 faces the user's skin on use for face cleansing. The cleaning article 100 consists of a base material composed of nonwoven materials composed of a mixture of natural cellulose-based fibers and synthetic fibers. This base material forms a wadding layer and is consolidated in that bonding of the fiber mixture is obtained by partial melting of the synthetic fibers.

In the case of a multilayer base material, the bonding of the plies can be achieved by calendering or laminating.

The base material of the cleaning article has a base ply having a grammage of preferably 80-300 g/m².

As FIG. 1 shows, a coating 112 composed of coating lines 114 is provided on the first side face 102 of the cleaning article 100. The coating 112 serves to remove skin flakes and makeup etc. in the case of face cleansing. The coating lines 114 are polymer-based. The material of the coating preferably has a Shore A hardness of 70-95. The coating lines are applied by means of a gravure method, wherein the cleaning article 100 is conducted between a gravure roll and an opposing roll. The width of the coating lines 114 in the present case is 0.5-1.0 mm. The height of the coating lines is preferably 0.2-0.4 mm, and so no unpleasant tactile effects occur on the skin by virtue of the coating pattern applied.

The coating shown in FIG. 1 has a multitude of individual patterns 120 formed by coating lines 114. In the case presented, preferably every individual pattern 120 is formed by groups of patterns 124, where the groups of patterns 124 here consist of four pattern elements 126. In the present groups of patterns, there are pattern elements 126 arranged encircling one another or else alongside one another and in contact with one another. No coating material has been applied between the individual pattern elements of every individual group of patterns that forms an individual pattern, i.e. there is an uncoated region 116 therein. In this way, the coating lines 114 overall achieve a degree of coverage on the first side face of about 10-25%. By virtue of the individual elements 120 as such, an overall area coverage of 30-40% of the first side face 102 is obtained, meaning that the clear areas outside the individual patterns 120, i.e. the outer uncoated regions 118 surrounding the individual patterns, take up about 60-70% of the first side face 102. In this way, the abrasiveness of the cleaning article 100 on the skin can be configured in an advantageous manner with simultaneously only minor impairment of the desired properties ascribed to the base material of the cleaning article, for example softness and/or absorption properties, which are not significantly influenced by the coating.

FIGS. 2 and 3 show further embodiments of cleaning articles 100. Thus, as shown in FIG. 2, the coating 112 on the first side face 102 may consist of a multitude of individual patterns 120 formed from coating lines 114, wherein the individual patterns 120 are in the form of groups of patterns 124 composed of at least 3 pattern elements 126, composed here of concentric circles. This gives rise to uncoated regions 116, 118 within the individual patterns and also outside the individual patterns. The individual patterns may also intersect with, overlap or touch one another, as apparent from FIG. 2.

With the coating 112, it is also possible for individual patterns 120 in rows that form words, formed here from coating lines 114, to be produced on the first side face of the cleaning article, as shown in FIG. 3. Here too, uncoated regions 116, 118 arise within and outside the individual patterns.

In addition, a coating in which the individual patterns 120 can intersect, overlap or touch one another, but each individual pattern in itself can still be seen and they especially cannot be connected by a continuous line running from one lateral edge of the cleaning article 122a to an opposite lateral edge of the cleaning article 122b, has the advantage that there are no preferential directions. The edges of the cleaning article 100 are considered in each case to be two opposite edges of the cleaning article 100. In this way, the abrasive properties can be improved in all directions.

Particular preference is given here to a coating in which, owing to the configuration of the individual patterns 120, at least one individual pattern 120, preferably at least 20% of the individual patterns 120, on the first side face, more preferably every individual pattern 120, has a section or region 128 that runs at right angles, i.e. with an angle 132 of 90°, to any direction 130 in the surface of the cleaning article 100, as shown in schematic form in FIG. 4a. In this way, any direction of movement can be faced by a proportion that runs at right angles thereto and hence has the optimal abrasion effect for this direction of movement.

The optimal manifestation of the advantages mentioned is achieved in that the individual patterns 120 are discrete from one another and especially do not merge into one another in such a way that the individual patterns 120 lose resolution in the entirety of the patterns, as is the case, for example, for the individual rhombuses or squares in a grid pattern.

Further preferred individual patterns are shown by FIGS. 4a-4c, it being possible both for different individual patterns to be combined with one another, as shown by FIGS. 4a-4b, and additionally also for the individual patterns to have a difference with regard to the formation of the coating lines both in terms of the height thereof and in terms of the width thereof. Furthermore, it is also conceivable to execute the coating lines not in a continuous, sustained manner but in an interrupted manner at least in sections, such as a dotted, dashed-and-dotted or dotted manner, as shown, for example, in FIG. 4a, provided that this does not lead to dissolution of the overall pattern in that the patterns as such can no longer be seen. The individual patterns formed from coating lines may also consist entirely of coating lines composed of a continuity of dots, as shown by way of example in FIG. 4c.

If an individual pattern 120 is composed of multiple pattern elements 126 as a group of patterns 124, these may, as shown in FIGS. 4a and 4b, encircle one another at a distance over the full circumference, but may also encircle one another such that there are points of contact. In addition, it is also possible that the individual pattern elements of a single pattern 120 are arranged to form regions of contact or intersection, as shown, for example, by FIG. 4c. The individual patterns according to FIGS. 4a to 4c may be formed analogously to FIG. 2 such that the individual patterns intersect, touch or overlap with one another.

The dynamic coefficient of friction of the coated side face of the cleaning article according to FIG. 1, measured in accordance with ASTM D 1894-01, is between 0.4 and 0.7.

The degrees of coverage on the first side face of a cleaning article are judged using an imaginary grid. The determination is elucidated in detail with reference to FIGS. 5a-c:

FIG. 5a shows, by way of example, a cleaning article 100 of rectangular dimensions by the top view of the first side face with a coating 112 distributed over the entire extent of the first side face. FIG. 5a is likewise intended to show, by way of example, a schematic detail from a sheetlike material 101 with a coating 112 applied thereto. It is possible by way of example to punch or cut cleaning articles 100 of smaller dimensions out of the sheetlike material 101, for example round cosmetic pads, as illustrated in FIG. 5b. The degrees of coverage are judged using an imaginary grid 200. The grid 200, formed from an arrangement of lines, wherein a first array 202 of parallel, equidistant lines is intersected by a second array 204 of parallel, equidistant lines to form right angles, consists of grid squares 206, each having an edge length of 7 mm. The grid may be placed in any desired manner over a cleaning article and its coating 112. Although the grid 200 here is provided with the arrays of lines at an angle of 45° to the edge 122 in the case of a rectangular cleaning article, no preferential direction for placement of the grid is intended for the judgement. The individual grid squares 206 and the degree of coverage present in each are ascertained. The evaluation is based on grid squares per 25 cm² having a first degree of coverage of at least 15% and grid squares per 25 cm² having a second degree of coverage of at least 8%. In the consideration of cleaning articles having a dimension other than 25 cm², the number of grid squares having a first degree of coverage of at least 15% ascertained on the first side face of the cleaning article and the number of grid squares having a second degree of coverage of at most 8% are correspondingly standardized to 25 cm². For the evaluation of the degree of coverage of the coating in relation to the respective grid squares, it is possible to use any desired evaluation methods, for example computer-assisted graphic and drawing programs. The judgement is shown by way of example for cleaning articles 100 in the configuration of round cosmetic pads as shown schematically in FIG. 5b: The grid 200 can be placed on as desired and the evaluation of the coating 112 on the cosmetic pads 100 is undertaken in relation to the grid squares of edge length 7 mm. In the evaluation, exclusively grid squares that are completely within the region of the cleaning article are used for the determination. Grid squares that are intersected by the boundary line (edge) 122 of the cleaning article 100, i.e. the cosmetic pad here, are disregarded for the consideration of the grid squares to be counted.

In the two cosmetic pads from FIG. 5b, the coating has 21 or 20 grid squares having a first degree of coverage of at least 15% and each coating has 10 grid squares having a second degree of coverage of at least 8%, as shown further in FIG. 5c. The fully filled grid squares here symbolize the first degree of coverage of at least 15% and the grid squares having a stripe pattern symbolize the second degree of coverage of at least 8%. This results in a coating having a coverage quotient, ascertained from the number of grid squares having the second degree of coverage divided by the number of grid squares having the first degree of coverage, of 0.5 for the cosmetic pads. This form of evaluation can be undertaken in any cleaning articles irrespective of their geometric shape or dimensions, for example round, oval, angular or any other shape.

1. A cleaning article (100) comprising an absorbent base material formed by a flat material (101) having a first side face (102) and a second side face, wherein a coating (112) that has been provided on at least the first side face (102) imparts elevated abrasiveness to the first side face (102) of the cleaning article compared to an uncoated first side face (102), and wherein the coating (112) comprises a multitude of coating lines (114) and wherein, when an imaginary grid (200) composed of parallel, equidistant lines running at right angles to one another with an edge length of 7 mm of each grid square (206) is placed over the first side face (102) of the cleaning article (100), the first side face (102) has a first degree of coverage of coating of at least 15% in the region of at least 5 grid squares (208) per 25 cm² and a second degree of coverage of coating of at most 8% in the region of at least 5 grid squares (210) per 25 cm².

2. The cleaning article (100) of claim 1, wherein the first side face (102) has a first degree of coverage of at least 15% in the region of at least 8, and at most 35 grid squares (208) per 25 cm².

3. The cleaning article (100) of claim 1, wherein the case of a coating (112) having at least 10 grid squares (208) per 25 cm² having the first degree of coverage of at least 15%, and at least 5 grid squares of these have a degree of coverage of at least 20%.

4. The cleaning article (100) of claim 1 wherein the grid squares (208) have an upper limit in the degree of coverage per 25 cm² of the first degree of coverage of at most 50%.

5. The cleaning article (100) of claim 1 wherein the coating (112) has a coverage quotient; ascertained from the number of grid squares (210) per 25 cm² of the second degree of coverage of not more than 8% divided by the number of grid squares (208) per 25 cm² of the first degree of coverage of at least 15%, of at least 0.1 and at most 3.0.

6. The cleaning article (100) of claim 1 wherein, the coating (112) has at least one region at least 2 and at most 30 grid squares (208) of the first degree of coverage of at least 15% are in a directly adjacent arrangement.

7. The cleaning article (100) of claim 1 wherein the coating (112) has or has been formed from a multitude of individual patterns (120) formed from coating lines (114).

8. The cleaning article (100) of claim 7, wherein at least one individual pattern (120) has at least one section (128) that runs at right angles to any direction in the first side face (102) and an imaginary tangent (134) applied thereto runs at right angles to any direction (130) in the first side face (102).

9. The cleaning article (100) of claim 7 at least one individual pattern (120) takes the form of a group of patterns (124) comprising at least two pattern elements (126) formed from coating lines (114).

10. The cleaning article (100) of claim 9, wherein a first pattern element (126) encircles at least regions of a second pattern element (126) or further pattern elements (126), and/or in that a first pattern element (126) is assigned to and arranged in contact with a second pattern element (126) or further pattern elements (126) alongside.

11. The cleaning article (100) of claim 7 wherein at least one individual pattern (120) on at least the first side face (102) is surrounded on all sides by an uncoated outer region.

12. The cleaning article (100) of claim 1 wherein the coating (112) covers the at least first side face (102) essentially over its entire extent.

13. The cleaning article, of claim 1 wherein the coating (112) may be distributed uniformly or non-uniformly across the cleaning article (100).

14. The cleaning article (100) of claim 1 wherein the first side face (102) has a degree of coverage by the coating (112) of at least 6% and at most 50%.

15. The cleaning article (100) of claim 1 wherein the coating lines (114) have a line width of at least 0.2 mm and at most 2.0 mm.

16. The cleaning article (100) of claim 1 wherein the coating lines (114) have a length corresponding to at least 5 times the width of the respective coating line (114).

17. The cleaning article (100) of claim 1 wherein the coating lines (114) have a height of at least 0.1 mm and of at most 0.8 mm.

18. The cleaning article (100) of claim 1 wherein the coating lines (114) take the form of continuous lines and/or lines that are interrupted at least in regions, where the interruption is not longer than 10 times the line width of the line adjacent to this site of interruption.

19. The cleaning article (100) of claim 1 wherein the coating (112) has a basis weight of at least 5 g/m² and of at most 50 g/m².

20. The cleaning article (100) of claim 1 wherein the coating (112) is polymer-based and formed from materials having a Shore A hardness of at least 30 and of at most 95.

21. The cleaning article (100) of claim 1 wherein the first side face (102) having the coating (112) has a dynamic coefficient of friction in accordance with ASTM D1894-01 of at least 0.2 and of not more than 1.5.

22. The cleaning article (100) of claim 6 further comprising wherein at least one region, in which at least 2 grid squares (210) of the second degree of coverage of not more than 8% are in a directly adjacent arrangement.
